(19) United States
(12) Patent Application Publication
(10) Pub. No.: US 2020/0370015 A1
(43) Pub. Date: Nov. 26, 2020

(54) METHOD FOR PRODUCING ERYTHROID PROGENITOR CELLS

(71) Applicants: ETABLISSEMENT FRANCAIS DU SANG, LA PLAINE SAINT DENIS CEDEX (FR); INSERM (INSTITUT NATIONAL DE LA SANTÉ ET DE LA RECHERCHE MÉDICALE), PARIS (FR); SORBONNE UNIVERSITÉ, PARIS (FR); UNIVÉRSITE PARIS-EST-CRÉTÉIL VAL DE MARNE, CRÉTEIL (FR)

(72) Inventors: LAURENCE GUYONNEAU-HARMAND, VILLEJUIF (FR); LO'I'C GARCON, PARIS (FR); FRÉDÉRIC AURADE, ISSY LES MOULINEAUX (FR); NICOLAS REBERGUE, SAINTE GENEVIEVE DES BOIS (FR); FRÉDÉRIC RELAIX, CHATENAY MALABRY (FR); LUC DOUAY, PARIS (FR)

(21) Appl. No.: 16/627,396

(22) PCT Filed: Jul. 2, 2018
(86) PCT No.: PCT/EP2018/067833

§ 371 Date: Dec. 30, 2019

(30) Foreign Application Priority Data
Jun. 30, 2017 (FR) 1756222

(51) Int. Cl. C12N 5/078

(52) U.S. Cl. CPC C12N 5/0641; C12N 2501/39; C12N 2501/999; C12N 2510/04; C12N 2506/02; C12N 2506/45; C12N 2502/45

(57) ABSTRACT

The present invention relates to a process for the in vitro production of erythroid progenitors comprising contacting hematopoietic stem cells, genetically modified or not, with a defined cell culture medium comprising a glucocorticoid hormone and an autophagy inducer.

The present invention falls within the field of medicine. More particularly, it relates to novel processes for the production of erythroid progenitors and red blood cells.

### TECHNOLOGICAL BACKGROUND OF THE INVENTION

Maintaining a constant supply of oxygen to tissues is essential for the survival of many living beings, especially humans. It is the red blood cells that carry oxygen within the body through the bloodstream. This transport is provided by hemoglobin, a protein specific to red blood cells that is capable of binding oxygen. When the red blood cells reach the tissues, the oxygen diffuses through the walls of the capillaries. The role of red blood cells is therefore essential.

Red blood cell transfusion is necessary in cases of emergency (bleeding) and pathology (blood diseases, cancers, etc.). In 2016, more than 100 million blood bags were collected worldwide and distributed to meet transfusion needs. Fifty percent of these products are distributed in rich countries, which represent only 15% of the global population. Transfusion issues in developing countries involve transfusion supply and safety, while in rich countries these issues are better controlled. However, immunological complications associated with chronic transfusions (alloimmunization) can lead to transfusion impasses involving the fate of patients.

To date, blood transfusions have been based exclusively on blood from donors.

In adults, red blood cell production, or erythropoiesis, occurs in the so-called hematopoietic bone marrow, which is present in flat bones and at the ends of long bones. In bone marrow, multipotent stem cells, called hematopoietic stem cells, differentiate successively into different types of erythroid progenitors (BFU-E, CFU-E, proerythroblasts, basophilic erythroblasts, polychromatophilic erythroblasts). When erythroblasts leave the bone marrow, they lose their nucleus to become reticulocytes and then mature red blood cells.

Differentiation media for hematopoietic stem cells allowing the in vitro production of red blood cells are known. However, current processes have critical flaws for large-scale production, such as a too rapid orientation toward red cell maturation, which significantly limits production yield, and differentiation into cells unable to perform enucleation steps correctly (Akimov S et al., 2005, Stem cells, 23(9): 1423-1433; Hirose S I et al., 2013, Stem Cell Reports, 1(6): 499-508; Huang X, 2013, Mol. Ther., 22(2): 451-463). Finally, other processes use co-cultures with feeder cells (Kurita R et al., 2013, PloS One, 8(3): e59890), which is both complex to implement and costly.

It appears that the development of novel processes for the in vitro production of red blood cells would make it possible to meet supply needs, to avoid emerging infectious risks and to avoid immunological complications.

The invention described here aims, among other things, to meet these needs.

### SUMMARY OF THE INVENTION

The inventors have demonstrated that the culture of hematopoietic stem cells in a medium containing dexamethasone, small-molecule enhancer of rapamycin-28 (SMER28), and optionally dimethyloxalylglycine (DMOG), not only differentiates these stem cells into erythroid progenitors, but also significantly amplifies this population of progenitors while preserving their ability to terminally differentiate into red blood cells. The erythroid progenitors thus obtained can be maintained in culture and amplified for more than 60 days without losing their ability to differentiate into mature enucleated cells, i.e., red blood cells.

According to a first aspect, the present invention relates to an in vitro process for the production of erythroid progenitors comprising contacting hematopoietic stem cells with a cell culture medium, preferably adapted to the nutritional requirements of hematopoietic stem cells and in particular adapted to the growth and/or differentiation of cells of the hematopoietic line, and comprising a glucocorticoid hormone and an autophagy inducer.

Preferably, the glucocorticoid hormone is selected from the group consisting of cortisone, hydrocortisone, prednisone, prednisolone, methylprednisolone, triamcinolone, paramethasone, betamethasone, dexamethasone, cortivazol, and derivatives and mixtures thereof. More particularly preferably, the glucocorticoid hormone is selected from the group consisting of prednisone, prednisolone and dexamethasone. Most particularly preferably, the glucocorticoid hormone is dexamethasone.

Preferably, the autophagy inducer is selected from the group consisting of small-molecule enhancer of rapamycin-28 (SMER-28), SMER-10 and SMER-18, and a combination thereof. More particularly preferably, the autophagy inducer is small-molecule enhancer of rapamycin-28 (SMER-28).

According to a particular embodiment, the culture medium also includes a hypoxia-inducible factor (HIF) pathway activator, preferably a prolyl hydroxylase inhibitor, and more preferably dimethyloxalylglycine (DMOG).

The hematopoietic stem cells are preferably obtained by differentiation of pluripotent stem cells, in particular embryonic stem cells (ES) or induced pluripotent stem cells (iPS), or are isolated from a sample of patient blood with or without mobilization, of umbilical cord blood or of placenta, or from a bone marrow sample or collection. Preferably, the hematopoietic stem cells are human hematopoietic stem cells. The hematopoietic stem cells may be genetically modified, in particular to overexpress one or more genes selected from the group consisting of human telomerase reverse transcriptase (HTERT), B lymphoma Mo-MLV insertion region 1 homolog (BMI1), c-MYC, 1-MYC and MYB. Preferably, the hematopoietic stem cells may be genetically modified to overexpress the HTERT gene or to overexpress the BMI1 gene. They can also be modified to overexpress the HTERT gene and the BMI1 gene.

The one or more genes selected from the group consisting of human telomerase reverse transcriptase (HTERT), B lymphoma Mo-MLV insertion region 1 homolog (BMI1), c-MYC, 1-MYC and MYB, are preferably placed under the control of one or more inducible promoters.

These hematopoietic stem cells may also be genetically modified to overexpress:
one or more core erythroid network (CEN) pathway transcription factors, preferably LIM domain only 2 (LMO2); and/or
one or more EPO-R/JAK2/STAT5/BCL-XL pathway genes, preferably B-cell lymphoma-extra large (BCL-XL).

Preferably, the hematopoietic stem cells are genetically modified to overexpress the BCL-XL gene or to overexpress the LMO2 gene.

The one or more genes selected from the group consisting of the EPO-R/JAK2/STAT5/BCL-XL pathway genes are preferably under the control of one or more constitutive promoters.

The one or more genes selected from the group consisting of the core erythroid network (CEN) pathway transcription factor genes are preferably under the control of one or more inducible promoters.

The hematopoietic stem cells may also be immortalized cells.

Preferably, the cells are cultured in the culture medium of the invention for at least 20 days, more preferably for at least 40 days, particularly preferably for at least 60 days.

In a second aspect, the invention further relates to the use of the cell culture medium according to the invention for the production and/or amplification of erythroid progenitors.

In a third aspect, the invention relates to genetically modified hematopoietic stem cells as described above and to the use of these hematopoietic stem cells for the in vitro production of erythroid progenitors and/or erythrocytes.

In a fourth aspect, the invention relates to an in vitro process for the production of erythrocytes comprising:
the production of erythroid progenitors according to the process of the invention; and
the induction of maturation of the erythroid progenitors,
and optionally, the recovery of the erythrocytes obtained.

Preferably, maturation of the erythroid progenitors is induced by culturing the erythroid progenitors in an erythrocyte differentiation medium.

In another aspect, the invention further relates to a cell culture medium, preferably adapted to the growth and/or differentiation of cells of the hematopoietic line, and comprising a glucocorticoid hormone, preferably dexamethasone, and an autophagy inducer, preferably SMER-28, and optionally a HIF pathway activator, preferably DMOG.

Preferably, the medium comprises a glucocorticoid hormone at a concentration between 0.01 mM and 0.1 mM, and/or an autophagy inducer at a concentration between 2 μM and 30 and/or a HIF pathway activator at a concentration between 75 and 350 μM.

The autophagy inducer is preferably selected from the group consisting of small-molecule enhancer of rapamycin-28 (SMER-28), SMER-10 and SMER-18, and a combination thereof, and more particularly preferably is SMER-28.

The glucocorticoid hormone is preferably selected from the group consisting of cortisone, hydrocortisone, prednisone, prednisolone, methylprednisolone, triamcinolone, paramethasone, betamethasone, dexamethasone, cortivazol, and derivatives and mixtures thereof, more particularly preferably is selected from the group consisting of prednisone, prednisolone and dexamethasone, and most particularly preferably is dexamethasone.

The hypoxia-inducible factor (HIF) pathway activator is preferably a prolyl hydroxylase (PHIS) inhibitor, and more preferably dimethyloxalylglycine (DMOG).

The culture medium may also include (i) transferrin, (ii) insulin, (iii) heparin, and (iv) serum, plasma, serum pool or platelet lysate, preferably platelet lysate, and optionally stem cell factor (SCF), EPO and/or IL-3.

The present invention further relates to the use of the cell culture medium according to the invention to produce and/or amplify erythroid progenitors.

In a fifth aspect, the invention further relates to a kit for the production of erythroid progenitors and/or erythrocytes comprising:
a culture medium according to the invention; and/or
genetically modified hematopoietic stem cells according to the invention; and
optionally, a guide containing instructions for using such a kit.

Finally, in a sixth aspect, the invention relates to the use of a kit according to the invention to produce erythroid progenitors and/or erythrocytes.

### DESCRIPTION OF THE DRAWINGS

FIG. 1: Expression profile of surface markers CD117 and CD235a on day 14, according to different culture protocols. A: protocol 4; B: protocol 3; C: protocol 1; D: protocol 2.

FIG. 2: Expression profile of surface markers CD117 and CD235a on day 24 of cells genetically modified to overexpress HTERT, BMI1 and LMO2, according to different culture protocols. A: protocol 4; B: protocol 1; C: protocol 2.

FIG. 3: Expression profile of surface markers CD117 and CD235a on day 24 of cells genetically modified to overexpress HTERT, BMI1 and BCL-XL, according to different culture protocols. A: protocol 4; B: protocol 1; C: protocol 2.

### DETAILED DESCRIPTION OF THE INVENTION

The inventors have demonstrated that the culture of hematopoietic stem cells in a medium comprising an autophagy inducer, namely small-molecule enhancer of rapamycin-28 (SMER28) and a glucocorticoid hormone, namely dexamethasone, significantly increases the production of erythroid progenitors compared with a control culture medium to which only dexamethasone is added. The erythroid progenitors thus obtained can be maintained in culture and amplified for more than 60 days. The inventors have also demonstrated that these erythroid progenitors are capable of effectively differentiating into red blood cells.

This in vitro culture process not only has the advantage of being simple and economical, but also opens the way for large-scale industrial production of erythroid progenitors and then red blood cells. This could reduce the risk of blood shortages or transfusion impasses, while providing optimal safety for transfused patients.

Thus, according to a first aspect, the present application relates to an in vitro process for the production of erythroid progenitors comprising the contacting of hematopoietic stem cells with a culture medium comprising an autophagy inducer and a glucocorticoid hormone. The purpose of this process is to induce the differentiation of hematopoietic stem cells into erythroid progenitors and to allow the amplification, i.e., the multiplication, of this population of progenitors while maintaining their ability to differentiate later into red blood cells. The process according to the invention is therefore a process for the production and amplification of erythroid progenitors.

As used here, the term “erythroid progenitors” refers to progenitor cells obtained by differentiation of hematopoietic stem cells during erythropoiesis. These progenitor cells are nucleated cells that have the ability to divide and later differentiate into red blood cells by enucleation. The erythroid progenitors are preferably selected from burst-forming units-E (BFU-E) characterized by the expression of markers CD117, CD34, CD41, CD71 and CXCR4, colony-forming units-E (CFU-E) characterized by the expression of markers CD117, CD34, CD36 and CD71, proerythroblasts characterized by the expression of markers CD117, CD71, CD36, and CD235a, basophilic erythroblasts characterized by the expression of markers CD117, CD71, CD36 and CD235a, and polychromatophilic erythroblasts characterized by the expression of markers CD36, CD71, CD235a, and mixtures thereof.

As used here, the term “hematopoietic stem cell” or “HSC” refers to multipotent stem cells capable of differentiating into blood cells and immune cells such as white blood cells, red blood cells and platelets. The hematopoietic stem cells express CD45, CD133, and/or CD34 antigens. Preferably, the hematopoietic stem cells express CD45 and CD34 antigens and, optionally, CD133 antigen.

According to the preferred embodiments, the HSCs are human HSCs.

These HSC can be obtained from different sources and according to procedures well known to the skilled person. In particular, they can be isolated from bone marrow, from cytapheresis, from whole blood or from umbilical cord blood (or placental blood), for example using an immunomagnetic system or a screening system for the presence of specific membrane receptors (for example CD133, CD45 and/or CD34).

As used here, the term “cytapheresis” refers to the removal of HSCs from the blood by apheresis. Apheresis is a technique for collecting certain blood components by extracorporeal blood circulation. The components to be collected are separated by centrifugation and extraction, while the components not collected are re-injected into the donor or patient (therapeutic apheresis).

HSCs can also be obtained by differentiation of pluripotent stem cells, in particular embryonic stem cells or induced pluripotent stem cells, preferably induced pluripotent stem cells. The techniques for differentiating pluripotent stem cells into HSCs are well known to the skilled person. Several protocols have been published, notably the protocol by Lengerke C et al. (2009, Ann NY Acad Sci, 1176:219-27) consisting of a 17-day differentiation through an intermediate stage of embryoid bodies and the combination of the following cytokines: SCF, Flt-3 ligand, IL-3, IL-6, G-CSF and BMP-4.

As used here, the term “embryonic stem cell” refers to cells derived from the internal cell mass of the blastocyst and which have the ability to lead to the formation of all tissues in the body (mesoderm, endoderm, ectoderm), including germ line cells. The pluripotency of embryonic stem cells can be assessed by the presence of markers such as transcription factors OCT4 and NANOG and surface markers such as SSEA3/4, Tra-1-60 and Tra-1-81. Embryonic stem cells can be obtained without destroying the embryo from which they are derived, for example by using the technique described by Chung et al. (Cell Stem Cell, 2008, 2(2): 113-117). In a particular embodiment, and for legal or ethical reasons, the embryonic stem cells are non-human embryonic stem cells. In another particular embodiment, the embryonic stem cells used in the invention are human embryonic stem cells, preferably obtained without destroying the embryo from which they are derived. The embryos used are preferably supernumerary embryos obtained during fertility treatment after obtaining regulatory and ethical approvals in accordance with the laws in force.

As used here, the term “induced pluripotent stem cell” (iPS) refers to pluripotent stem cells obtained by genetic reprogramming of differentiated somatic cells, and having a morphology and potential for self-renewal and pluripotency that are partially similar to embryonic stem cells. These cells are particularly positive for pluripotency markers, including alkaline phosphatase staining and expression of the proteins NANOG, SOX2, OCT4 and SSEA3/4. The processes for obtaining induced pluripotent stem cells are well known to the skilled person and are described in particular in articles by Yu et al. (Science, 2007, 318 (5858): 1917-1920), Takahashi et al. (Cell, 2007, 131(5): 861-872) and Nakagawa et al. (Nat Biotechnol, 2008, 26(1): 101-106).

The HSCs used in the process according to the invention may also be genetically modified HSCs in order to increase their ability to engage in erythropoiesis and/or their amplification capacity. Thus, according to certain embodiments, the process according to the invention may include the genetic modification of HSCs in order to increase their ability to engage in erythropoiesis and/or their amplification capacity. The methods for genetically modifying these cells are well known to the skilled person and involve, for example, the introduction of transgenes into the cell genome via retroviruses or lentiviruses or any other form of gene or protein transfer.

According to an embodiment, the amplification capacity of these HSCs is improved by overexpressing one or more genes selected from the group consisting of human telomerase reverse transcriptase (HTERT), B lymphoma Mo-MLV insertion region 1 homolog (BMI1), c-MYC, 1-MYC and MYB.

According to a particular embodiment, the HSCs are genetically modified to overexpress HTERT.

According to another specific embodiment, the HSCs are genetically modified to overexpress HTERT and one or more genes selected from the group consisting of B lymphoma Mo-MLV insertion region 1 homolog (BMI1), c-MYC, 1-MYC and MYB.

According to a preferred embodiment, the HSCs are genetically modified to overexpress HTERT and/or BMI1, preferably HTERT and BMI1.

The ability of HSCs to engage in the erythropoiesis pathway can be improved by overexpressing one or more genes involved in the EPO-R/JAK2/STAT5/BCL-XL pathway and/or in the core erythroid network (CEN) pathway.

As used here, the term “EPO-R/JAK2/STAT5/BCL-XL pathway” refers to a cellular signaling pathway whose key proteins are the erythropoietin (EPO) receptor, Janus kinase 2 (JAK2), signal transducer and activator of transcription 5 (STAT5) and B-cell lymphoma-extra large (BCL-XL). EPO is essential for erythropoiesis, it promotes erythroid involvement and cell survival. The binding of EPO to its membrane receptor causes the dimerization of EPO-R which, when activated, in turn induces JAK2. JAK2 then phosphorylates the tyrosine residues of the cytoplasmic tail of EPO-R. These phosphotyrosines allow interaction with proteins containing a Src homology 2 (SH2) domain, which results in the activation of different signaling pathways, the main one being the STAT5 pathway. STAT5 is first dimerized and then phosphorylated, which leads to its translocation into the nucleus where it activates the transcription of different genes including genes involved in cell proliferation and erythroid differentiation.

According to an embodiment, the HSCs are genetically modified to overexpress one or more genes of the EPO-R/JAK2/STAT5P/BCL-XL pathway, preferably one or more genes selected from the group consisting of the genes encoding EPO-R, JAK2, STAT5P, BCL-XL and BCL-2, and a combination thereof.

According to a particular embodiment, the HSCs are genetically modified to overexpress a gene encoding BCL-XL.

As used here, the term “CEN pathway” refers to a group of transcription factors essential for establishing or maintaining the identity of erythrocytes.

According to an embodiment, the HSCs are genetically modified to overexpress one or more CEN pathway genes, preferably one or more genes selected from the group consisting of the genes encoding GATA1 (transcription factor belonging to the family of zinc-finger proteins binding to the DNA sequence “GATA”), TAL bHLH transcription factor 1 (TAL1), Kruppel-like factor 1 (KLF1), LIM domain binding 1 (LDB1), LIM domain only 2 (LMO2) and stem cell leukemia (SCL), and a combination thereof.

According to a particular embodiment, the HSCs are genetically modified to overexpress a gene encoding LMO2.

According to a particular embodiment, the HSCs used in the process according to the invention are genetically modified to overexpress:

(i) one or more genes selected from the group consisting of the genes encoding HTERT, BMI1, c-MYC, 1-MYC and MYB, and a combination thereof, preferably HTERT and/or BMI1, and more particularly preferably HTERT and BMI1; and

(ii) one or more EPO-R/JAK2/STAT5/BCL-XL pathway genes, preferably selected from the group consisting of the genes encoding EPO-R, JAK2, STAT5, BCL-XL and BCL-2, and a combination thereof, and more particularly preferably BCL-XL; and/or

(iii) one or more CEN pathway genes, preferably selected from the group consisting of the genes encoding GATA1, TAL1, KLF1, LDB1, LMO2 and SCL, and a combination thereof, and more particularly preferably LMO2.

According to another particular embodiment, the HSCs used in the process according to the invention are genetically modified to overexpress:

(i) one or more genes selected from the group consisting of the genes encoding HTERT, BMI1, c-MYC, 1-MYC and MYB, and a combination thereof, preferably HTERT and/or BMI1, and more particularly preferably HTERT and BMI1; and

(ii) one or more EPO-R/JAK2/STAT5/BCL-XL pathway genes, preferably selected from the group consisting of the genes encoding EPO-R, JAK2, STAT5P, BCL-XL and BCL-2, and a combination thereof, and more particularly preferably BCL-XL; and

(iii) one or more CEN pathway genes, preferably selected from the group consisting of the genes encoding GATA1, TAL1, KLF1, LDB1, LMO2 and SCL, and a combination thereof, and more particularly preferably LMO2.

According to a preferred embodiment, the HSCs are genetically modified to overexpress

(i) the gene encoding HTERT, and optionally the gene encoding BMI1, and

(ii) the gene encoding LMO2 and/or the gene encoding BCL-XL, preferably the gene encoding BCL-XL.

In particular, the HSCs may be genetically modified to overexpress the gene encoding HTERT and the gene encoding BCL-XL, and optionally the gene encoding BMI1.

According to another preferred embodiment, the HSCs are genetically modified to overexpress

(i) the genes encoding HTERT and BMI1, and

(ii) the gene encoding LMO2 and/or the gene encoding BCL-XL.

In particular, the HSCs may be genetically modified to overexpress

(i) the genes encoding HTERT and BMI1 and the gene encoding LMO2, or

(ii) the genes encoding HTERT and BMI1 and the gene encoding BCL-XL.

As used here, the term “overexpression,” “overexpress” or “overexpressing” refers to the level of expression of a gene in a genetically modified cell that is higher than the level of expression of the same gene in the non-genetically modified cell. When the cell does not express the gene in question before the genetic modification but expresses it after modification, this term may be replaced by “expression,” “express” or “expressing.”

The overexpression of a gene in an HSC may be achieved by any technique known to the skilled person, in particular by introduction into the HSC of a nucleic acid comprising the one or more genes to be overexpressed, or several nucleic acids each comprising one of the genes to be overexpressed. The one or more nucleic acids can thus be arranged on the same construct or in separate constructs. They can be introduced into the HSC by any method known to the skilled person, in particular by viral transduction, microinjection, transfection, electroporation and biolistics.

As used here, the term “construct” refers to an expression cassette or an expression vector.

In an expression cassette, the one or more genes to be overexpressed are operably linked to the sequences necessary for their expression. In particular, they may be under the control of a promoter allowing their expression in an HSC. Generally, an expression cassette includes, or consists of, a promoter to initiate transcription, one or more genes, and a transcription terminator. The expression “operably linked” indicates that the elements are combined so that the expression of the coding sequence is under the control of a transcriptional promoter. Typically, the promoter's sequence is placed upstream (5′) of the one or more genes of interest. Spacer sequences may be present between the regulatory elements and the gene, provided they do not prevent expression by translation of the encoded protein. The expression cassette may also include at least one “enhancer” activating sequence operably linked to the promoter.

An expression vector includes one or more nucleic acids or expression cassettes as described. This expression vector can be used to transform a host cell and allow the expression of the nucleic acid of interest in said cell. Vectors can be constructed by conventional molecular biology techniques, well known to the skilled person.

Advantageously, the expression vector includes regulatory elements allowing the expression of the nucleic acid of interest. These elements may include, for example, transcription promoters, transcription activators, terminator sequences, start and stop codons. The methods for selecting these elements are well known to the skilled person.

The vector may be circular or linear, single- or double-stranded. It is advantageously selected from plasmids, phages, phagemids, viruses, cosmids and artificial chromosomes. Preferably, the vector is a viral vector.

The one or more genes to be overexpressed can be placed under the control of identical or different constitutive or inducible promoters, whether or not they are present on the same nucleic acid.

The HSC can be transiently or stably transformed/transfected and the one or more nucleic acids, cassettes or vectors can be contained in the cell as an episome or integrated into the HSC genome. They can be inserted into the eukaryotic cell genome in identical or distinct regions.

There are many techniques well known to the skilled person allowing the stable or transient expression of genes of interest. In particular, genes of interest can be integrated into a cell's genome using a knock-in technique using a targeted expression system, in particular the CRISPR-Cas9 system (see for example Platt et al. Cell. 2014 Oct. 9; 159(2):440-55 or Lo et al. Biotechniques. 2017 Apr. 1; 62(4):165-174). This technique, which allows a single copy of one or more genes of interest to be inserted into the cell genome at a predetermined locus, is based on the transfection of one or more vectors allowing the coordinated expression of a gene encoding the Cas9 nuclease and a guide RNA (gRNA) specific to the locus where the one or more genes are to be inserted. Said one or more genes of interest or a cassette containing said one or more genes of interest are inserted as a result of the repair of the break generated by Cas9.

As used in the present application, the term “guide RNA” or “gRNA” refers to an RNA molecule capable of interacting with Cas9 to guide it to a target chromosome region.

Each gRNA can include two regions:
a first region (commonly referred to as the “SDS” region), at the 5′ end of the gRNA, which is complementary to the target chromosome region and mimics the crRNA of the endogenous CRISPR system, and
a second region (commonly known as the “handle” region), at the 3′ end of the gRNA, which mimics the base pairing interactions between the trans-activating crRNA (tracrRNA) and the endogenous CRISPR system crRNA and has a double-stranded stem-loop structure with an essentially single-stranded sequence at the 3′ end. This second region is essential for gRNA-Cas9 binding.

The gene of interest or the cassette comprising the one or more genes of interest is flanked by homologous sequences of the break site targeted by the gRNA, allowing the gene or cassette to be inserted through break repair by homologous recombination.

Examples of promoters allowing constitutive expression include, but are not limited to, pEF1 alpha long, pCMV and pCAG.

An inducible expression system that can be used in the present invention is the Tet-On system, based on the use of the tetracycline transactivator protein (tTA), which is created by fusing the tetracycline repressor (TetR) protein present in Escherichia coli bacteria with the activating domain of the VP16 protein present in herpes virus. The rtTA protein is able to bind to DNA on a specific TetO operating sequence only if it is bound to a tetracycline. Several repeating TetO sequences are placed under the control of a promoter such as the EF1 alpha long promoter. TetO sequences coupled to the promoter are called a tetracycline response element (TRE) and respond to tetracycline transactivator protein (tTA) binding by causing an increase in the expression of the gene under the promoter's control.

When several genes are overexpressed, they may be placed under the control of the same promoter or of several promoters.

According to a particular embodiment, the HSCs are genetically modified to overexpress one or more genes selected from the group consisting of the genes encoding HTERT, BMI1, c-MYC, 1-MYC and MYB, and a combination thereof, preferably HTERT and/or BMI1, and more particularly preferably HTERT and BMI1, and this or these genes are placed under the control of one or more inducible promoters.

According to a particular embodiment, the HSCs are genetically modified to overexpress one or more CEN pathway genes, preferably selected from the group consisting of the genes encoding GATA1, TAL1, KLF1, LDB1, LMO2 and SCL, and a combination thereof, and more particularly preferably LMO2, and this or these genes are placed under the control of one or more inducible promoters.

According to a particular embodiment, the HSCs are genetically modified to overexpress one or more EPO-R/JAK2/STAT5/BCL-XL pathway genes, preferably selected from the group consisting of the genes encoding EPO-R, JAK2, STAT5P, BCL-XL and BCL-2, and a combination thereof, and more particularly preferably BCL-XL, and this or these genes are placed under the control of one or more constitutive promoters.

According to a preferred embodiment, the HSCs are genetically modified to overexpress the gene encoding HTERT under the control of an inducible promoter and the gene encoding BCL-XL under the control of a constitutive promoter.

According to another preferred embodiment, the HSCs are genetically modified to overexpress the genes encoding HTERT, BMI1 and LMO2 under the control of one or more inducible promoters.

According to another preferred embodiment, the HSCs are genetically modified to overexpress the genes encoding HTERT and BMI1 under the control of one or more inducible promoters and the gene encoding BCL-XL under the control of a constitutive promoter.

Alternatively, or in addition to the genetic modifications described above, the HSCs as used in the process according to the invention may also be genetically modified to contain a suicide gene, for example the HSV-TK gene or the Casp9 gene, under the control of an inducible promoter. As used here, the term “suicide gene” refers to any gene whose expression causes the death of the division-capable cell that expresses it, in the presence or absence of an additional molecule (medicament or other), depending on the suicide gene considered. By way of example, cell death of a cell expressing the HSV-TK gene or the Casp9 gene is obtained, respectively, by adding ganciclovir or AP1003.

According to certain embodiments, the HSCs as used in the process according to the invention are immortalized HSCs. These immortalized cells may further be genetically modified as described above.

Immortalized HSCs can be obtained from an immortalized cell line established from malignant cells.

Preferably, immortalized HSCs are obtained from an immortalized cell line established from non-malignant cells, for example from iPS (Kurita et al. PLoS ONE, 2013, 8, e59890), from umbilical cord blood cells (Kurita et al. supra; Huang, X. et al. Mol. Ther. 2014, 22, 451-463), from embryonic stem cells (Hirose, S. et al. Stem Cell Rep. 2013, 1, 499-508), or from HSCs isolated from bone marrow, from cytapheresis or from whole peripheral blood (Trakarnsanga et al., 2017, Nature Communications, Vol. 8, 14750).

HSCs can be immortalized by any technique known to the skilled person, notably by transduction with a lentiviral vector carrying human papillomavirus type 16 oncogenes E6 and E7 (HPV16 E6/E7) (Akimov et al. Stem Cells. 2005 October; 23(9): 1423-1433; Trakarnsanga et al. supra). Optionally, the HSCs may also be transduced with a lentiviral vector carrying the gene encoding HTERT (Akimov et al., supra).

According to a preferred embodiment, the immortalized HSCs used in the process according to the invention contain a suicide gene allowing their elimination after induction of the maturation of erythroid progenitors into mature enucleated erythrocytes.

The process according to the invention includes contacting the HSCs as described above with a glucocorticoid hormone and an autophagy inducer, and more particularly with a culture medium adapted to the growth and/or differentiation of cells of the hematopoietic line and comprising a glucocorticoid hormone and an autophagy inducer.

As used here, the terms “autophagy,” “autolysis” and “autophagocytosis” are equivalent and can be used interchangeably. Autophagy refers to the degradation of part of the cell's cytoplasm by its own lysosomes.

Autophagy is a physiological process that helps to eliminate certain proteins (viral, malformed, etc.) and damaged organelles. This process can also be involved in the elimination of intracellular pathogens. Several signaling pathways detect different types of cellular stress, ranging from nutrient deprivation to microbial invasion, and converge to regulate autophagy. As used here, the term “autophagy inducer” refers to a molecule capable of inducing autophagy in a cell.

In particular, autophagy inducers may be mTOR pathway inhibitors such as metformin, rapamycin, perifosine, everolimus, resveratrol or tamoxifen, autophagosome formation activators such as the compound MG-132 (a 26S proteasome inhibitor), the compound SAHA (a pan-histone deacetylase inhibitor), trichostatin A or valproic acid, or small molecules acting independently of the mTOR pathway such as SMER-28, SMER-10 or SMER 18.

According to a particular embodiment, the autophagy inducer is an inducer acting independently of the mTOR pathway, preferably selected from the group consisting of small-molecule enhancer of rapamycin-28 (SMER-28), SMER 10 and SMER 18, and a combination thereof.

According to a preferred embodiment, the autophagy inducer is SMER-28.

As used here, the terms “glucocorticoid hormone,” “glucocorticoid,” “corticosteroid,” or “corticoid” are equivalent and can be used interchangeably. These terms refer to natural or synthetic steroid hormones with a pregnane nucleus and an action on protein and carbohydrate metabolism.

According to an embodiment, the glucocorticoid hormone is selected from the group consisting of cortisone, hydrocortisone, prednisone, prednisolone, methylprednisolone, triamcinolone, paramethasone, betamethasone, dexamethasone, cortivazol, and derivatives and mixtures thereof.

According to a particular embodiment, the glucocorticoid hormone is a synthetic hormone, preferably selected from the group consisting of prednisone, prednisolone, methylprednisolone, triamcinolone, paramethasone, betamethasone, dexamethasone, cortivazol, and derivatives and mixtures thereof. According to a preferred embodiment, the glucocorticoid hormone is selected from the group consisting of prednisolone, methylprednisolone, dexamethasone, and derivatives and mixtures thereof, preferably from the group consisting of prednisolone, methylprednisolone and dexamethasone.

According to a particularly preferred embodiment, the glucocorticoid hormone is dexamethasone.

According to a particular embodiment, the autophagy inducer is selected from the group consisting of SMER-28, SMER 10 and SMER 18, preferably is SMER-28, and the glucocorticoid hormone is selected from the group consisting of prednisolone, methylprednisolone and dexamethasone, preferably is dexamethasone.

According to a very particular embodiment, the autophagy inducer is SMER-28 and the glucocorticoid hormone is dexamethasone.

Optionally, the HSCs may also be contacted with an activator of the HIF pathway. As used here, the term “HIF pathway” refers to the signaling pathway initiated by hypoxia-inducible factor (HIF) which stimulates EPO secretion and thus activates the EPO-R/JAK2/STAT5/BCL-XL pathway.

Preferably, the HIF pathway activator according to the invention is a prolyl hydroxylase (PHIS) inhibitor.

As used here, the terms “prolyl hydroxylase (PHIS)” and “procollagen-proline dioxygenase” are equivalent and can be used interchangeably. Prolyl hydroxylase is a hydroxylation enzyme of HIF on its prolyl residues. When hydroxylated, HIF is inhibited. The specific inhibitors of prolyl hydroxylase are therefore molecules capable of inhibiting prolyl hydroxylase and thus activating the HIF pathway which in turn activates the EPO-R/JAK2/STAT5/BCL-XL pathway.

Preferably, the prolyl hydroxylase inhibitor is selected from the group consisting of dimethyloxalylglycine (DMOG), N-oxalylglycine (NOG), desferrioxamine (DFO), FG-4383, F-0041, FG-2216, FG-4592, 5956711, ethyl-3,4 dihydroxy-benzoate (EDHB), TM6089, TM655, TM6008, 8-hydroxyquinoline, and derivatives thereof.

Most particularly preferably, the HIF pathway activator is DMOG.

During the implementation of the process according to the invention, the HSCs are cultured in a culture medium adapted to the growth and/or differentiation of the cells of the hematopoietic line. Many culture media adapted to the nutritional requirements of HSCs are known to the skilled person and commercially available, such as StemSpan SFEM (STEMCELL Technologies) preferably supplemented with stem cell factor (SCF), erythropoietin (EPO), and lipids or StemMACS HSC Expansion Media XF, human” (Invitrogen).

Preferably, the HSCs are cultured at a concentration between 200 and 10000 cells/mL, preferably between 500 and 2000 cells/mL, and more preferably around 1000 cells/mL.

The culture medium is preferably changed about every 3 days so that the cells do not exceed a concentration of about 4000000 cells/mL.

The HSCs may be contacted with the glucocorticoid hormone and the autophagy inducer on the first day of culture or after several days of culture, for example after 10 to 15 days.

Preferably, the HSCs are contacted simultaneously with the glucocorticoid hormone and the autophagy inducer.

Alternatively, the HSCs may be first contacted with the glucocorticoid hormone and then with the autophagy inducer, or vice versa. The addition of the second compound may take place, for example, a few hours after contact with the first compound.

Preferably, the HSCs are contacted simultaneously with the glucocorticoid hormone and the autophagy inducer. More particularly preferably, the HSCs are brought into simultaneous contact with the glucocorticoid hormone and the autophagy inducer from the first day of culture.

Contact may be achieved by adding the glucocorticoid hormone and/or autophagy inducer to the HSC culture medium or by placing the HSCs in a culture medium containing the glucocorticoid hormone and/or the autophagy inducer.

The concentrations of the glucocorticoid hormone and autophagy inducer may be constant or vary throughout the culture or contact.

Preferably, when the culture medium includes glucocorticoid hormone, the latter is present at a concentration between 0.001 mM and 10 mM, preferably between 0.01 mM and 1 mM, more preferably between 0.01 mM and 0.5 mM, and particularly preferably between 0.02 mM and 0.1 mM.

According to a particular embodiment, when the culture medium includes the glucocorticoid hormone, the latter is present at a concentration of about 0.1 mM.

Preferably, when the culture medium includes the autophagy inducer, the latter is present in the culture medium at a concentration between 0.1 μM and 100 μM, preferably between 0.5 μM and 50 μM, more preferably between 1 μM and 30 μM, and particularly preferably between 2 μM and 30 μM. Alternatively, when the culture medium includes the autophagy inducer, the latter may be present in the culture medium at a concentration between 10 μM and 30 μM.

According to a particular embodiment, when the culture medium includes the autophagy inducer, the latter is present in the culture medium at a concentration of about 2 μM.

As used here, the term “about” refers to a range of values of ±5% of the specified value, preferably ±2% of the specified value. For example, “about 20” includes 20±5%, or 19 to 21.

Similarly, in embodiments where the HSCs are placed in the presence of a HIF pathway activator, the concentration of the activator may be constant or may vary throughout the culture or contact.

Preferably, when the culture medium includes a HIF pathway activator, preferably DMOG, the latter is present in the culture medium at a concentration between 1 μM and 1000 μM, preferably between 10 μM and 500 μM, more preferably between 50 μM and 400 μM, and particularly preferably between 75 μM and 350 μM.

According to a particular embodiment, the HSCs are placed in the presence of a glucocorticoid hormone, preferably dexamethasone, and an autophagy inducer, preferably SMER28, after 10 to 15 days of culture. Preferably, according to this embodiment, the concentration of the glucocorticoid hormone is between 0.01 mM and 0.5 mM, and more particularly preferably between 0.02 mM and 0.1 mM, and the concentration of the autophagy inducer is between 10 μM and 30 μM.

According to another particular embodiment, the HSCs are placed in the presence of a glucocorticoid hormone, preferably dexamethasone, and an autophagy inducer, preferably SMER28, from the first day of culture or before 10 days of culture. Preferably, according to this embodiment, the concentration of the glucocorticoid hormone is between 0.01 mM and 0.5 mM, preferably about 0.1 mM, and the concentration of the autophagy inducer is between 2 μM and 30 μM, preferably about 2 μM.

The HSCs are preferably maintained in a culture medium comprising a glucocorticoid hormone and an autophagy inducer, and optionally a HIF pathway activator, for at least 10 days. More particularly preferably, the HSCs are maintained in a culture medium containing a glucocorticoid hormone and an autophagy inducer for at least 20, 30, 40, 50 or 60 days.

It is understood that, during this step, the cell culture includes not only HSCs but also erythroid progenitors.

The inventors have demonstrated that the use of a culture medium containing a glucocorticoid hormone and an autophagy inducer significantly amplified the production of erythroid progenitors, which do not engage in the terminal erythroid differentiation pathway. Thus, according to the process of the invention, the HSCs can be cultured in the culture medium containing a glucocorticoid hormone and an autophagy inducer for at least 60, 70, 80, 90 or 100 days. Preferably, the HSCs are cultured in the culture medium containing a glucocorticoid hormone and an autophagy inducer for a maximum of 70, 80, 90 or 100 days.

The duration of HSC culture and the contact time with a glucocorticoid hormone and an autophagy inducer, and optionally a HIF pathway activator, can be easily defined by the skilled person by assessing the proportion of erythroid progenitors present in the cell population. Preferably, the HSCs are contacted with a glucocorticoid hormone and an autophagy inducer until a population comprising at least 90% erythroid progenitors, preferably at least 95% erythroid progenitors, more preferably at least 99% erythroid progenitors, is obtained.

The culture may be maintained until markers of maturation into mature erythrocytes appear. Preferably, the culture is stopped when the cells no longer amplify and/or less than 10%, preferably less than 5%, of them express the membrane receptor CD117. Alternatively, the culture may be stopped when at least 90%, preferably at least 95%, of the cells in culture have reached the polychromatophilic erythroblast stage or are at a more advanced stage of differentiation.

According to certain embodiments, the process may include alternating phases of culture during which the culture medium includes a glucocorticoid hormone and an autophagy inducer, and optionally a HIF pathway activator, and phases of culture during which the culture medium does not include glucocorticoid hormone, autophagy inducer, and/or HIF pathway activator.

The glucocorticoid hormone, the autophagy inducer and the HIF pathway activator may be added or removed from the culture medium simultaneously or sequentially.

The techniques for modifying the composition of a culture medium are well known to the skilled person. In particular, the addition of a molecule or the increase in its concentration may be done directly in the pre-existing culture medium and the removal of a molecule or decrease in its concentration may be done by centrifuging the cells and resuspending them in a new culture medium or by diluting the culture medium.

The process according to the invention may further include a step of recovering the erythroid progenitors obtained. This step may be carried out by any technique known to the skilled person, in particular by centrifugation and removal of the culture medium. The process of the invention may also include a cell sorting step, in particular a cell selection step based on the expression of the marker CD117. CD117+ cells can be selected to prolong the amplification of erythroid progenitor cells. Conversely, CD117− cells can be selected to produce red blood cells.

The process according to the invention may also include a step of washing the erythroid progenitors obtained/recovered. This step may be carried out by any technique known to the skilled person, in particular by a succession of centrifugation and resuspension steps.

According to a particular aspect, the invention also relates to a population of erythroid progenitors obtained by the process of the invention.

According to another aspect, the invention also relates to the use of erythroid progenitors obtained by the process according to the invention for the production of erythrocytes.

As used here, the terms “red blood cell,” “mature red blood cell,” “red blood cell,” “red corpuscle,” “erythrocyte” and “mature erythrocyte” are equivalent and can be used interchangeably. The term “erythrocyte” refers to an enucleated cell with markers characteristic of erythrocyte maturation. They express in particular glycophorin A (CD235a) but do not express the marker CD36.

The present invention thus relates to an in vitro process for the production of erythrocytes comprising:
the production of erythroid progenitors according to the process of the invention described above; and
induction of maturation of the erythroid progenitors.

The embodiments described above for the process for the production of erythroid progenitors according to the invention are also considered in this aspect.

Maturation of erythroid progenitors may include expression of erythrocyte maturation markers such as CD235a and by enucleation.

The process of the invention may also include a cell sorting step, in particular a CD117⁻ cell selection step.

Maturation of the progenitors can be induced by any method known to the skilled person. In particular, maturation can be induced by culturing erythroid progenitors in an erythrocyte differentiation medium, for example a medium supplemented with erythropoietin and optionally SMER28. Preferably, maturation is induced by culturing erythroid progenitors in a medium that does not include stem cell factor (SCF) or dexamethasone and is supplemented with erythropoietin (about 2.5 IU/mL) and optionally SMER28 (about 2.5 μM). Alternatively, maturation is induced by placing the cells in a serum-free culture medium. Preferably, progenitor maturation is induced at high cell concentrations, for example above 5,000,000 cells/mL of culture.

According to an embodiment, the process for the production of erythrocytes according to the invention further includes a step of eliminating nucleated cells. This step results in a homogeneous population comprising only mature erythrocytes.

According to a particular embodiment in which the HSCs used in the process for the production of erythroid progenitors according to the invention include an inducible suicide gene, this step of eliminating nucleated cells can be performed by inducing the expression of this suicide gene.

The process for the production of erythrocytes may further include a step of recovering the erythrocytes obtained. This step may be carried out by any technique known to the skilled person, in particular by filtration, centrifugation and removal of the culture medium.

The process according to the invention may also include a step of washing the erythrocytes obtained/recovered. This step may be carried out by any technique known to the skilled person, in particular by a succession of filtration, centrifugation and resuspension steps.

According to another aspect, the present invention also relates to a population of erythrocytes obtained by the process of the invention.

According to another aspect, the present invention also relates to a pharmaceutical composition comprising erythroid progenitors obtained by the process of the invention and a pharmaceutically acceptable carrier.

The invention also relates to erythroid progenitors according to the invention, or a pharmaceutical composition comprising erythroid progenitors according to the invention, for use as a hematopoietic graft. As used here, the term “hematopoietic graft” refers to a set of cells intended to be administered to the bone marrow of a subject and capable of producing erythrocytes.

The invention also relates to erythroid progenitors according to the invention, or a pharmaceutical composition comprising erythroid progenitors according to the invention, for use in the treatment of anemia.

As used here, the term “anemia” refers to an abnormal blood count characterized by an abnormally low level of healthy red blood cells and a decrease in circulating hemoglobin below normal values for the subject's age. By way of illustration, anemia is generally characterized by a hemoglobin level of less than 13 g/dL for a man, less than 12 g/dL for a woman, less than 11 g/dL for a child and less than 14 g/dL for a newborn.

Anemia can have various and multiple causes. Examples of anemia include, but are not limited to, anemia related to bleeding (for example bleeding caused by trauma or surgery), anemia induced by drug therapy (for example chemotherapy) or exposure to toxic substances (for example lipolytic agents, oxidizing agents, lead, venom or poisons), hemolytic anemia caused by an inherited disorder of the erythrocyte membrane (for example inherited spherocytosis, inherited elliptocytosis or inherited pyropoikilocytosis), hemolytic anemia caused by an acquired disorder of the erythrocyte membrane (for example paroxysmal nocturnal hemoglobinuria or acanthocytosis, autoimmune hemolytic anemia (for example transfusion accident), anemia caused by an infectious agent (for example malaria, Babesia or Bartonella infection, trypanosomiasis, visceral leishmaniasis, septicemia or CMV infection), hereditary or acquired sideroblastic anemia, anemia caused by bone marrow failure (for example bone marrow depression, vitamin B12 deficiency, myelodysplasia or bone marrow disease invasion by malignant hematopathy (leukemia, lymphoma, metastasis)), or anemia related to sickle cell disease or to thalassemic syndrome. Preferably, the anemia is associated with sickle cell disease or with thalassemic syndrome.

The invention further relates to a method for treating a patient suffering from anemia comprising administering to said patient a therapeutically effective amount of erythroid progenitors obtained by the process of the invention, or a pharmaceutical composition comprising erythroid progenitors obtained according to the invention.

The invention also relates to the use of erythroid progenitors obtained according to the invention, or a pharmaceutical composition comprising said progenitors, for the preparation of a medicament, in particular a biological medicament, for the treatment of anemia.

As used here, the term “biological medicament” refers to a medicinal product whose active substance is produced by or extracted from a biological source.

Preferably, in this aspect, the erythroid progenitors are obtained from non-genetically modified HSCs.

As used here, the terms “subject” and “patient” are equivalent and can be used interchangeably. These terms refer preferably to an animal, in particular a mammal, most particularly preferably a human, in particular a fetus, a newborn, a child, a teenager, an adult or an elderly person. As used here, the term “fetus” refers to an intrauterine developmental stage of more than 8 weeks of pregnancy, the term “newborn” refers to a human being under 12 months of age, the term “child” refers to a human being aged 1 to 12 years, the term “adult” refers to a human being aged 12 to 60 years and the term “elderly person” refers to a human being aged 60 years or older.

The patient is preferably a patient who has failed a transfusion, is polytransfused, or has a rare blood group. According to a preferred embodiment, this patient suffers from anemia, in particular anemia related to sickle cell disease or to thalassemic syndrome.

As used here, the term “transfusion failure” refers to a transfusion that is ineffective and/or induces pathological complications in the patient.

A red cell transfusion may be considered ineffective when, 24 hours after a transfusion of red cell concentrates (RCC), the transfusion efficiency is less than 80%.

Erythrocyte transfusion efficiency (ETE) is calculated by the formula:

$$ETE = \frac{( HB level after transfusion ) - ( Hb level before transfusion )}{amount of HB transfused / TBV of the patient} \times 100$$

The minimum amount of HB transfused is 40 g, the average observed is 50 g. TBV refers to total blood volume.

Pathological complications associated with transfusion failure may be the consequence of an immunizing transfusion (transfusion alloimmunization) that may occur years later and compromise the patient's future transfusions. Indeed, during a new transfusion, prior immunization may either cause a direct hemolytic danger (if the antibodies are present at a sufficient titer), or, more often, cause delayed hemolysis (if the antibodies are present at a low titer or even not detectable serologically during the new transfusion).

As used here, the term “polytransfused” refers to a patient who has undergone several blood transfusions and/or a patient who has already had a blood transfusion and is to receive another.

As used here, the term “rare blood group” refers to a blood group whose frequency in the French and/or European and/or global population is less than 1/250 and/or to a blood group whose patient who carries it cannot be transfused with O− blood. Rare blood presents supply difficulties.

In the field of veterinary applications, the subject of the invention may be a non-human animal, preferably a pet or a farm animal, for example selected from the group consisting of dogs, cats, cattle, sheep, rabbits, pigs, goats, horses, rodents, non-human primates and poultry.

As used here, the term “treatment” refers to any action aimed at improving or eliminating symptoms, slowing the progression of the disease, stopping the progression of the disease or eliminating the disease. This term refers more specifically to an increase in the level of healthy red blood cells and circulating hemoglobin, preferably to reach normal values for the subject's age. This term includes both preventive and curative treatment. The term “therapeutically effective amount” as used here refers to an amount sufficient to have an effect on at least one symptom of the condition, and more specifically to increase the level of healthy red blood cells and circulating hemoglobin in the treated subject.

As used here, the terms “pharmaceutically acceptable carrier” and “pharmaceutically acceptable support” are equivalent and refer to any substance other than an active principle present in a pharmaceutical composition. Its addition is intended in particular to facilitate the preservation and administration of cells, without modifying their properties. The pharmaceutically acceptable carrier used for the formulation of compositions containing erythrocytes or erythrocyte progenitors according to the invention may be, for example, selected from the group consisting of saline, PBS solution with human serum albumin added, and mixtures thereof, or any other saline solution having an osmolarity suitable for the preservation of erythrocytes and/or progenitors and, preferably, which can be directly administered to the subject. For the formulation of compositions containing erythrocytes, a saline-adenine-glucose-mannitol (SAGM) medium may also be used as a pharmaceutically acceptable carrier, alone or in combination with the other pharmaceutically acceptable carriers listed above.

Preferably, the administration of progenitors, or graft, is performed at the patient's bone marrow or by intravenous injection.

According to a preferred embodiment, the hematopoietic stem cells used to produce erythroid progenitors are derived from a donor sample or from cells obtained from a donor and the erythroid progenitors are intended to be transplanted into a recipient patient. The donor and the recipient may be the same individual (autologous graft) or different individuals (allogeneic graft). Preferably, the donor and the recipient are the same individual.

According to another aspect, the invention relates to a pharmaceutical composition or a medicament, in particular a biological medicament, comprising erythrocytes obtained by the process of the invention and a pharmaceutically acceptable carrier.

The invention also relates to erythrocytes obtained by the process of the invention or a pharmaceutical composition comprising erythrocytes obtained according to the invention, for the transfusion of patients suffering from anemia, i.e., requiring a supply of erythrocytes.

The invention also relates to a method for treating a patient suffering from anemia, i.e., requiring a supply of erythrocytes, comprising administering a therapeutically effective amount of erythrocytes obtained by the process of the invention, or a pharmaceutical composition comprising erythrocytes according to the invention.

The invention also relates to erythrocytes obtained by the process of the invention or a pharmaceutical composition comprising erythrocytes obtained according to the invention, for use in the treatment of anemia.

Preferably the anemia is an anemia related to sickle cell disease or to thalassemic syndrome.

Preferably, the patients are transfusion failures, are polytransfused or have rare blood.

In the context of personalized medicine, the erythrocytes to be administered or administered to the patient are obtained according to an in vitro process for the production of red blood cells comprising:
obtaining HSCs from a sample from said patient;
obtaining erythroid progenitors from said HSCs according to the process of the invention; and
obtaining erythrocytes from said erythroid progenitors according to the process of the invention.

The HSCs can be obtained from a sample of the patient's blood or bone marrow. Alternatively, the HSCs can be obtained from iPSC obtained by genetic reprogramming of differentiated somatic cells obtained from the patient.

The HSCs may optionally be genetically modified as described above.

According to another aspect, the present invention further relates to HSCs genetically modified to overexpress:

(i) one or more genes selected from the group consisting of the genes encoding HTERT, BMI1, c-MYC, 1-MYC and MYB, and a combination thereof, preferably HTERT and/or BMI1, and more particularly preferably HTERT and BMI1; and (ii) one or more EPO-R/JAK2/STAT5/BCL-XL pathway genes, preferably selected from the group consisting of the genes encoding EPO-R, JAK2, STAT5P, BCL-XL and BCL-2, and a combination thereof, and more particularly preferably BCL-XL; and/or

(iii) one or more CEN pathway genes, preferably selected from the group consisting of the genes encoding GATA1, TAL1, KLF1, LDB1, LMO2 and SCL, and a combination thereof, and more particularly preferably LMO2.

The embodiments concerning HSCs used in the process for the production of erythroid progenitors are also considered in this aspect.

According to a preferred embodiment, the HSCs are genetically modified to overexpress the gene encoding HTERT under the control of an inducible promoter and the gene encoding BCL-XL under the control of a constitutive promoter.

According to another preferred embodiment, the HSCs are genetically modified to overexpress the genes encoding HTERT, BMI1 and LMO2 under the control of one or more inducible promoters.

In another preferred embodiment, the HSCs are genetically modified to overexpress the genes encoding HTERT and BMI1 under the control of one or more inducible promoters and the gene encoding BCL-XL under the control of a constitutive promoter.

The genetically modified HSCs may also contain a suicide gene as described above or be immortalized.

The present invention also relates to the use of genetically modified HSCs according to the invention for the production, preferably in vitro, of erythroid progenitors and/or erythrocytes, in particular according to the processes of the invention described above.

According to still another aspect, the present invention relates to a cell culture medium adapted to the nutritional requirements of HSCs, and in particular adapted to the growth and/or differentiation of cells of the hematopoietic line, and comprising a glucocorticoid hormone and an autophagy inducer, and optionally a HIF pathway activator.

The embodiments concerning the medium comprising a glucocorticoid hormone and an autophagy inducer, and optionally a HIF pathway activator, used in the process for the production of erythroid progenitors, are also considered in this aspect.

The glucocorticoid hormone and the autophagy inducer are as defined above for the process according to the invention. Preferably, the glucocorticoid hormone is dexamethasone and/or the autophagy inducer is SMER28 and/or the HIF pathway activator is DMOG.

The base of the cell culture medium adapted to the growth and/or differentiation of cells of the hematopoietic line may be any base known by the skilled person to meet the needs of HSCs and/or erythroid progenitors.

As used here, the term “growth” refers to the multiplication of cells. The term “differentiation” refers to the acquisition by cultured cells of characteristics that were not present in the cells initially used to inoculate the medium. In the present case, this term refers to the acquisition of characteristics of erythroid progenitors. A medium adapted to the growth and/or differentiation of cells of the hematopoietic line is therefore a medium allowing the differentiation of HSCs into erythroid progenitors as well as the multiplication of HSCs and of erythroid progenitors. This term should not be confused with “maturation,” which here defines the process by which erythroid progenitors will become red blood cells and which involves, in particular, the enucleation of cells. The medium adapted to the growth and/or differentiation of cells of the hematopoietic line therefore preferably does not contain any compounds that induce this maturation.

The base of the cell culture medium may include Iscove's modified Dulbecco's medium (IMDM) or an equivalent medium adapted to the nutritional requirements of HSCs (for example StemSpan SFEM (STEMCELL Technologies) or StemMACS HSC Expansion Media XF, human, Invitrogen), supplemented with insulin, transferrin, stem cell factor (SCF), heparin, IL-3, EPO, growth factors, and/or serum, plasma, platelet lysate and/or serum pool. Compounds added to the medium are preferably human compounds obtained by recombinant or purification techniques. The concentrations of these different compounds are easily determined by the skilled person on the basis of the suppliers' recommendations or general knowledge of the field.

As used here, the term “serum pool” refers to a mixture of human AB plasmas (most often a mixture of more than 100 different plasmas) that are viro-attenuated. To do this, AB plasmas from transfusion centers are mixed, viro-attenuated and finally aliquoted. The serum pool can then be used fresh or kept frozen.

As used here, the term “platelet lysate” refers to a product rich in growth factors that is obtained as a result of the lysis of platelet concentrates. To do this, platelet concentrates from transfusion centers can be mixed before being lysed. There are different lysis methods well known to the skilled person, in particular ultrasound lysis, the use of solvents and/or detergents, or cryolysis. Platelet concentrates are preferably lysed by cryolysis. Cryolysis consists of freeze/thaw cycles, usually two cycles, causing the platelets to rupture and the growth factors they contain to be released into the plasma. Optionally, lysis may be followed by centrifugation and/or filtration. The platelet lysate may then be used fresh or kept frozen.

Preferably, the base of the medium according to the invention is an IMDM as defined above or an equivalent medium, supplemented with:
transferrin, preferably human transferrin, at a concentration between about 200 μg/mL and about 400 μg/mL, preferably between about 300 μg/mL and about 350 μg/mL, more preferably at a concentration of about 330 μg/mL; and/or
insulin, preferably human insulin, at a concentration between about 1 μg/mL and about 50 μg/mL, preferably between about 5 μg/mL and about 20 μg/mL, more preferably at a concentration of about 10 μg/mL; and/or
serum, plasma or serum pool, preferably human, at a concentration between about 1% and about 10%, preferably between about 3% and about 7%, more preferably at a concentration of about 5%, and/or a platelet lysate, preferably of human origin, at a concentration between about 0.05% and about 0.5%, preferably between about 0.1% and about 0.5%, more preferably at a concentration of about 0.3%; and/or
heparin, preferably human heparin, at a concentration between about 0.5 U/mL and about 10 U/mL, preferably between about 1 U/mL and about 5 U/mL, more preferably at a concentration of about 3 U/mL.

Optionally, in particular for a culture medium adapted to the growth and/or differentiation of cells of the hematopoietic line, the medium may also be supplemented with:
IL-3, preferably human IL-3, at a concentration between about 1 ng/mL and about 20 ng/mL, preferably between about 3 ng/mL and about 7 ng/mL, more preferably at a concentration of about 5 ng/mL;
SCF, preferably human, at a concentration between about 10 ng/mL and about 200 ng/mL, preferably between about 50 ng/mL and about 150 ng/mL, more preferably at a concentration of about 100 ng/mL; and/or
EPO, preferably human, at a concentration between about 0.5 IU/mL and about 10 IU/mL, preferably between about 1 IU/mL and about 5 IU/mL, more preferably at a concentration of about 2 IU/mL.

In a particular embodiment, the base of the culture medium according to the invention is preferably an IMDM as defined above or an equivalent medium, and includes transferrin, insulin, heparin, and serum, plasma, serum pool or platelet lysate, preferably transferrin, insulin, heparin and serum pool or platelet lysate, more particularly preferred, transferrin, insulin, heparin and platelet lysate. These compounds are preferably used in concentrations as described above.

In a preferred embodiment, this medium further includes IL-3, SCF and EPO, preferably at concentrations as described above.

Alternatively, the medium may include SCF and EPO, preferably at concentrations as described above.

According to a particular embodiment, the base of the medium according to the invention includes:
transferrin, preferably human transferrin, at a concentration between 300 μg/mL and 350 μg/mL;
insulin, preferably human insulin, at a concentration between 5 μg/mL and 20 μg/mL;
serum, plasma or serum pool, preferably human, at a concentration between 3% and 7%, and/or platelet lysate, preferably of human origin, at a concentration between 0.1% and 0.5%; and
heparin, preferably human heparin, at a concentration between 1 U/mL and 5 U/mL, and optionally,
IL-3, preferably human IL-3, at a concentration between 3 ng/mL and 7 ng/mL;
SCF, preferably human, at a concentration between 50 ng/mL and 150 ng/mL; and/or
EPO, preferably human, at a concentration between 1 IU/mL and 5 IU/mL.

The medium according to the invention comprises, added to this base, (i) an autophagy inducer, preferably SMER-28, (ii) a glucocorticoid hormone, preferably dexamethasone, and optionally (iii) a HIF pathway activator, preferably DMOG.

According to a particular embodiment, the medium according to the invention includes, added to this base:
a glucocorticoid hormone, preferably dexamethasone, at a concentration between 0.001 mM and 10 mM, preferably between 0.002 mM and 1 mM, more preferably between 0.005 mM and 0.5 mM, and particularly preferably between 0.01 mM and 0.1 mM; and
an autophagy inducer, preferably SMER-28, at a concentration between 0.1 μM and 100 μM, preferably between 0.5 μM and 50 μM, more preferably between 1 μM and 30 and particularly preferably between 2 μM and 30 μM; and
optionally, a HIF pathway activator, preferably DMOG, at a concentration between 1 μM and 1000 preferably between 10 μM and 500 more preferably between 50 μM and 400 and particularly preferably between 75 μM and 350 μM.

In a particular embodiment, the medium according to the invention comprises between 0.005 mM and 0.5 mM glucocorticoid hormone, preferably dexamethasone, between 0.5 μM and 50 μM autophagy inducer, preferably SMER-28, and optionally between 50 μM and 400 μM HIF pathway activator, preferably DMOG.

In another particular embodiment, the medium according to the invention comprises between 0.01 mM and 0.1 mM glucocorticoid hormone, preferably dexamethasone, between 2 μM and 30 μM autophagy inducer, preferably SMER-28, and optionally between 75 μM and 350 μM HIF pathway activator, preferably DMOG.

Preferably, the medium according to the invention does not include serum of non-human origin (for example fetal calf serum), thrombopoietin, vascular endothelial growth factor (VEGF), IL-6, bone morphogenetic protein (BMP), FLT3-ligand and/or hydrocortisone.

The present invention also relates to the use of the cell culture medium according to the invention and as described above for the production and/or amplification of erythroid progenitors and/or the production of erythrocytes, in particular according to the processes of the invention described above, and more particularly to stimulate the differentiation of HSCs into erythroid progenitors and/or to amplify erythroid progenitors.

In another aspect, the present invention further relates to a kit comprising:
a cell culture medium according to the invention; and/or
genetically modified HSCs according to the invention or genetic constructs to obtain the genetically modified HSCs according to the invention; and
optionally, a guide containing instructions for using such a kit.

The present invention also relates to the use of a kit according to the invention to produce erythroid progenitors and/or erythrocytes, in particular according to the processes of the invention described above.

All references cited in this application, including journal articles or abstracts, published patent applications, granted patents or any other reference, are fully incorporated here by reference, which includes all results, tables, figures and texts presented in these references.

Although having different meanings, the terms “comprising,” “having,” “containing” and “consisting of” may be replaced by each other throughout the description of the invention.

Other features and advantages of the invention will appear more clearly upon reading the following illustrative and non-limiting examples.

### Examples

### Example 1: Amplification of Erythroid Progenitors from HSCs Derived from Umbilical Cord Blood and from Cytapheresis

### Materials and Methods

Medium Used:

IMDM (Biochrom), supplemented with transferrin (330 μg/mL), insulin (10 μg/mL), 5% AB serum pool (EFS) and heparin (3 U/mL). From day 0 to day 7, the medium was supplemented with IL-3 (5 ng/mL), SCF (100 ng/mL) and EPO (2 IU/mL). From day 7 until the end of the amplification of erythroblastic progenitors, the medium was supplemented with SCF (100 ng/mL) and EPO (2 IU/mL).

HSCs:

HSCs are obtained after magnetic separation using CD34+ beads according to the protocol provided by Milenyi (see CD34 MicroBead Kit, human, from Miltenyi Biotec).

Cell Maintenance:

On day 0 the cells were seeded at a concentration of 10,000 cells/mL, on day 4 the cells were diluted 1/5 in new medium, on day 7 the cells were washed and cultured at 100,000 cells/mL in new medium. On day 11 the cells were diluted to 100,000 cells/mL and seeded in a new medium. On day 14 the cells were reseeded at 300,000 cells/mL in a new medium. On day 18 the cells were diluted to 0.5 million/mL. From day 21 onwards, the cells were systematically returned to 1 million/mL on each maintenance day (i.e., twice per week).

Culture Protocols:

Protocol 1: From day 0 to day 11 the cells are cultured in the basal medium. On day 11 the medium used is supplemented with 253.9 μM DMOG. On day 14, 333 μM DMOG, 0.02 mM dexamethasone (DEX) and 30 μM SMER28 are added to the medium used. The medium used on day 18 is supplemented with 0.09 mM DEX and 20.1 μM SMER28. On day 21, 0.10 mM DEX and 24.5 μM SMER28 were added to the medium used, while 0.10 mM DEX and 17.4 μM SMER28 were added to that of day 25. Finally, from day 28, the medium was systematically supplemented with 0.10 mM DEX and 13.8 μM SMER28.

Protocol 2: from day 0 to the end of the culture the medium is supplemented with 0.1 mM DEX and 2.27 μM SMER28.

Protocol 3: from day 0 to the end of the culture the medium is supplemented with 0.1 mM DEX.

Protocol 4: this protocol is the control protocol; it was carried out with the basal medium without adding any factor.

Flow Cytometry:

A sample of 100,000 cells is collected, washed, and then exposed to the CD235a and CD117 antibodies, according to the supplier's instructions. After 30 minutes at room temperature and in the dark, the cells are washed twice with PBS. The cells are then ready for cytometric analysis.

The CD235a antibody specifically recognizes glycophorin A which indicates mature erythroid involvement;

The CD117 antibody specifically recognizes the receptor c-kit (i.e., stem cell factor receptor) that indicates the immaturity, the strain and the self-renewal ability of cells

Cell Counts:

The cells are diluted to a tenth in trypan blue solution, allowing cell mortality to be assessed if necessary.

### Results

TABLE 1
Cell count after culture according to different protocols

| | Protocol 1 | Protocol 2 | Protocol 3 | Protocol 4 |
|---|---|---|---|---|
| amplification of erythroid | 9.00 · 10⁷ | 9.19 · 10⁷ | 2.47 · 10⁷ | 3.47 · 10⁵ |
| progenitors/CD34+ from | | | | |
| cytapheresis on day 58 | | | | |
| amplification of erythroid | 7.73 · 10¹⁰ | 3.64 · 10¹⁰ | ND | 2.94 · 10⁷ |
| progenitors/CD34+ from | | | | |
| umbilical cord blood | | | | |
| on day 78 | | | | |

Protocols 1 and 2 allow exponential amplification of erythroblasts. The amplification obtained with protocols 1 and 2 is also substantially higher than that obtained with dexamethasone alone (see Table 1). It is also interesting to note that protocols 1 and 2 allow amplification of erythroid progenitors from CD34+ cells derived from cytapheresis or from umbilical cord blood.

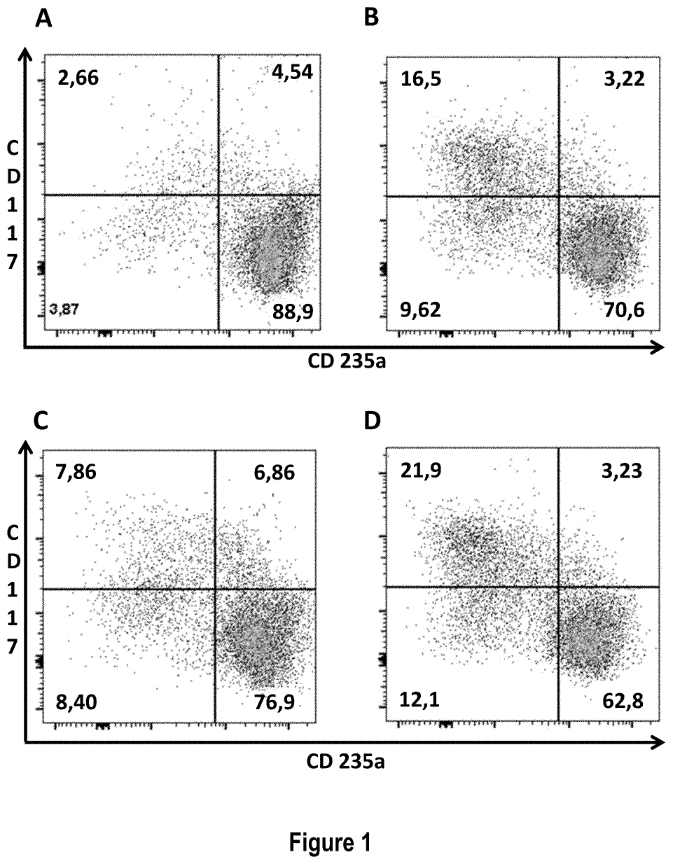

The C-KIT marker persists much longer under these conditions than under the control conditions (see FIG. 1); this membrane marker indicates the youthfulness of the cells and their ability to proliferate.

This work has demonstrated that the culture of human hematopoietic stem cells in a culture medium according to the invention makes it possible to obtain:
cells with total erythroid involvement;
an exponential enrichment and amplification of erythroid progenitors.

In particular, amplification may last up to 78 days.

### Example 2: Amplification of Erythroid Progenitors from Engineered HSCs Derived from Cytapheresis Inducibly Overexpressing HTERT, BMI1 and Constitutively Overexpressing BCL-XL or Inducibly Overexpressing HTERT, BMI1 and LMO2

### Materials and Methods

Medium Used:

IMDM (Biochrom), supplemented with transferrin (330 μg/mL), insulin (10 μg/mL), 5% AB serum pool (EFS) and heparin (3 U/mL). From day 0 to day 7, the medium was supplemented with IL-3 (5 ng/mL), SCF (100 ng/mL) and EPO (2 IU/mL). From day 7 until the end of the amplification of erythroblastic progenitors, the medium was supplemented with SCF (100 ng/mL) and EPO (2 IU/mL).

HSCs:

HSCs are obtained from cytapheresis after magnetic separation using Miltenyi CD34+ beads.

Cell Maintenance:

On day 0 the cells were seeded at a concentration of 100,000 cells/mL, for two successive infections with the HTERT BMI1 lentiviral supernatant and the BCL-XL retroviral supernatant or the HTERT BMI1 lentiviral supernatant and the LMO2 lentiviral supernatant; on day 3 the cells were washed 3 times and reseeded at a concentration of 10,000 cells/mL, on day 4 the cells were diluted to 1/5 in new medium. On day 7 the cells were washed and cultured at 100,000 cells/mL in new medium. On day 11 the cells were diluted to 100,000 cells/mL and seeded in a new medium. On day 14 the cells were reseeded to 300,000 cells/mL in a new medium. On day 18 the cells were diluted to 0.5 million/mL. From day 21 onwards, the cells were systematically reset to 1 million/mL each day of maintenance (i.e., twice per week).

Culture Protocols:

Protocol 1: From day 0 to day 11 the cells are cultured in the basal medium. On day 11 the medium used is supplemented with 253.9 μM DMOG. On day 14, 333 μM DMOG, 0.02 mM dexamethasone (DEX) and 30 μM SMER28 are added to the medium used. The medium used on day 18 is supplemented with 0.09 mM DEX and 20.1 μM SMER28. On day 21, 0.10 mM DEX and 24.5 μM SMER28 were added to the medium used, while 0.10 mM DEX and 17.4 μM SMER28 were added on day 25. Finally, from day 28 the medium was systematically supplemented with 0.10 mM DEX and 13.8 μM SMER28.

Protocol 2: from day 0 to the end of the culture the medium is supplemented with 0.1 mM DEX and 2.27 μM SMER28.

Protocol 3: from day 0 to the end of the culture the medium is supplemented with 0.1 mM DEX.

Protocol 4: this protocol is the control protocol; it was carried out with the basal medium without adding any factor.

### Results

TABLE 2
Cell count on day 65 after culture according to different protocols

| | amplification of erythroid progenitors/CD34+ | | | |
|---|---|---|---|---|
| | Protocol 1 | Protocol 2 | Protocol 3 | Protocol 4 |
| CD34+ HTERT | 1.06 · 10⁸ | 1.82 · 10⁸ | 5.12 · 10⁶ | 8.50 · 10⁵ |
| BMI1 LMO2 | | | | |
| CD34+ HTERT | 1.59 · 10⁶ | 1.06 · 10⁹ | 5.76 · 10⁵ | 6.92 · 10⁵ |
| BMI1 BCL-XL | | | | |

Protocols 1 and 2 allow exponential amplification of erythroblasts with both engineered HSC models (see Table 2). The amplification obtained with protocols 1 and 2 is also substantially higher than that obtained with dexamethasone alone.

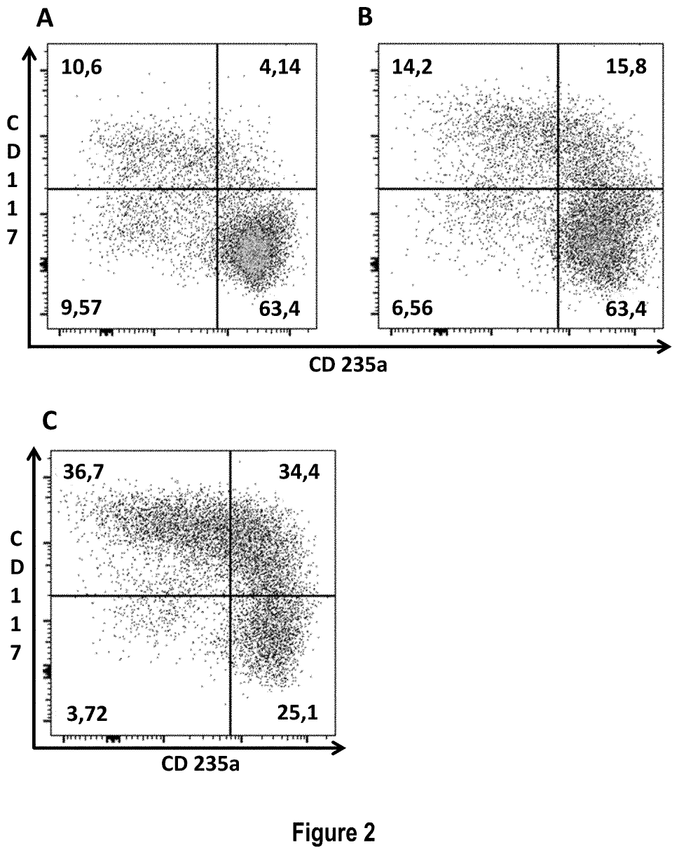

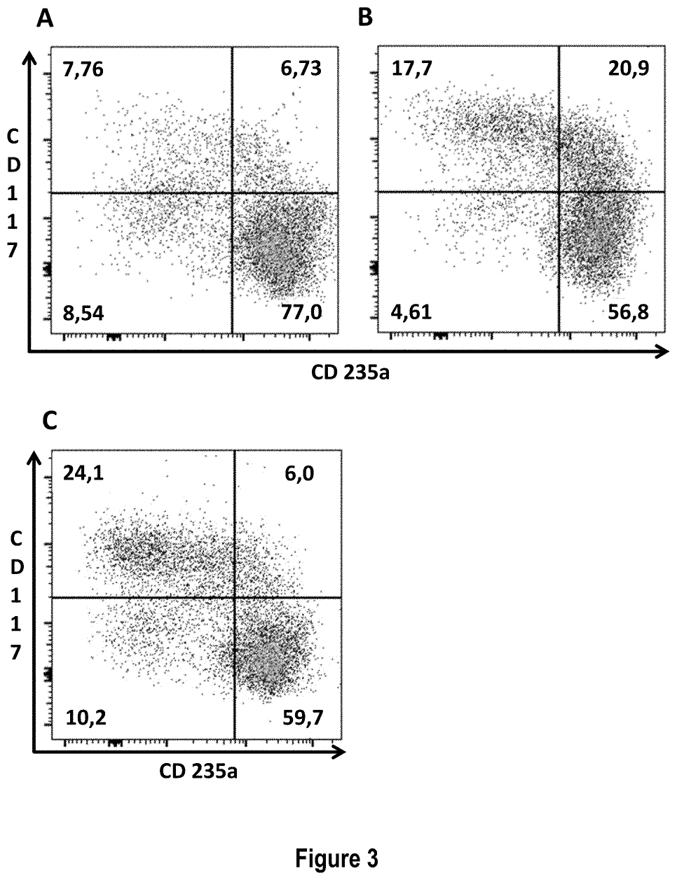

The C-KIT marker persists under these conditions much longer than in the controls; this membrane marker indicates the youthfulness of the cells and their ability to proliferate (see FIGS. 2 and 3). Protocol 2 allows for superior amplification with both types of engineered HSCs.

These amplifications are remarkable because the engineered cells are CD34⁺ derived from cytapheresis which are known for their low amplification rate (compared with CD34+ from umbilical cord blood).

With these engineered cell protocols, CD34⁺ cells derived from cytapheresis acquire greater amplification capacities than CD34⁺ cells derived from umbilical cord blood.

1-40. (canceled)

41. In vitro process for the production of erythroid progenitors comprising contacting hematopoietic stem cells with a cell culture medium comprising an autophagy inducer and a glucocorticoid hormone.

42. The process according to claim 41, wherein the autophagy inducer is selected from the group consisting of small-molecule enhancer of rapamycin-28 (SMER-28), SMER-10 and SMER-18, and a combination thereof.

43. The process according to claim 41, wherein the glucocorticoid hormone is selected from the group consisting of cortisone, hydrocortisone, prednisone, prednisolone, methylprednisolone, triamcinolone, paramethasone, betamethasone, dexamethasone, cortivazol, and derivatives and mixtures thereof.

44. The process according to claim 41, wherein the culture medium further comprises a hypoxia-inducible factor (HIF) pathway activator, a prolyl hydroxylase (PHIS) inhibitor, or dimethyloxalylglycine (DMOG).

45. The process according to claim 41, wherein the hematopoietic stem cells are obtained by differentiation of pluripotent stem cells, embryonic stem cells (ES), induced pluripotent stem cells (iPS), or are isolated from a sample of patient blood, isolated from umbilical cord or placental blood, or isolated from a bone marrow sample.

46. The process according to claim 41, wherein the hematopoietic stem cells are human hematopoietic stem cells.

47. The process according to claim 41, wherein the hematopoietic stem cells are genetically modified to overexpress one or more genes selected from the group consisting of human telomerase reverse transcriptase (HTERT), B lymphoma Mo-MLV insertion region 1 homolog (BMI1), c-MYC, 1-MYC and MYB.

48. The process according to claim 47, wherein the one or more genes selected from the group consisting of human telomerase reverse transcriptase (HTERT), B lymphoma Mo-MLV insertion region 1 homolog (BMI1), c-MYC, 1-MYC and MYB, are placed under the control of one or more inducible promoters.

49. The process according to claim 47, wherein the hematopoietic stem cells are further genetically modified to overexpress:
one or more core erythroid network (CEN) pathway transcription factors; and/or
one or more EPO-R/JAK2/STAT5/BCL-XL pathway genes.

50. The process according to claim 49, wherein the one or more genes selected from the group consisting of EPO-R/JAK2/STAT5/BCL-XL pathway genes are placed under the control of one or more constitutive promoters.

51. The process according to claim 49, wherein the one or more genes selected from the group consisting of the core erythroid network (CEN) pathway transcription factor genes are placed under the control of one or more inducible promoters.

52. The process according to claim 41, wherein the hematopoietic stem cells are immortalized and/or include a suicide gene.

53. A genetically modified hematopoietic stem cell comprising one or more genes selected from the group consisting of human telomerase reverse transcriptase (HTERT), B lymphoma Mo-MLV insertion region 1 homolog (BMI1), c-MYC, 1-MYC and MYB, said genetically modified hematopoietic stem cells overexpressing said one or more genes.

54. In vitro process for the production of erythrocytes comprising:
the production of erythroid progenitors according to the process of claim 41; and
the induction of maturation of the erythroid progenitors,
and optionally the recovery of the erythrocytes obtained.

55. Cell culture medium adapted for the growth and/or differentiation of cells of the hematopoietic line and comprising a glucocorticoid hormone, an autophagy inducer, and, optionally, a HIF pathway activator.

56. The cell culture medium according to claim 55, comprising
a glucocorticoid hormone at a concentration between 0.01 mM and 0.1 mM, and/or
an autophagy inducer at a concentration between 2 μM and 30 μM, and/or
a HIF pathway activator at a concentration between 75 μM and 350 μM.

57. The cell culture medium according to claim 55, wherein the culture medium further comprises a hypoxia-inducible factor (HIF) pathway activator, a prolyl hydroxylase (PHIS) inhibitor, or dimethyloxalylglycine (DMOG).

58. The cell culture medium according to claim 55, further comprising (i) transferrin, (ii) insulin, (iii) heparin, and (iv) serum, plasma, serum pool or platelet lysate.

59. The cell culture medium according to claim 55, further comprising stem cell factor (SCF), EPO, and, optionally, IL-3.

60. Kit for the production of erythroid progenitors and/or erythrocytes comprising:
a cell culture medium as defined in claim 55; and/or
genetically modified hematopoietic stem cells comprising one or more genes selected from the group consisting of human telomerase reverse transcriptase (HTERT), B lymphoma Mo-MLV insertion region 1 homolog (BMI1), c-MYC, 1-MYC and MYB, said genetically modified hematopoietic stem cells overexpressing said one or more genes; and
optionally, a guide containing instructions for using such a kit.
